(19) United States
(12) Patent Application Publication
(10) Pub. No.: US 2012/0245707 A1
(43) Pub. Date: Sep. 27, 2012

(54) Locking device, in particular for prostheses

(75) Inventors: Rudolf Osgyan, Regensburg (DE); Helmut Tischler, Regensburg (DE); Dieter Birkner, Turkenfeld (DE); Thomas Krafczyke, Emmering (DE)

(73) Assignee: STREIFENEDER ORTHO.PRODUCTION GMBH, Emmering (DE)

(21) Appl. No.: 13/419,060

(22) Filed: Mar. 13, 2012

(30) Foreign Application Priority Data
Mar. 14, 2011 (DE) 102011013815.3-35

(51) Int. Cl. A61F 2/60

(52) U.S. Cl. 623/32

(57) ABSTRACT

The object of providing a device which renders it possible to allow an artificial knee joint at least temporarily a further degree of freedom so as to enable a natural course of movement is solved by the locking device according to the invention for use in a prosthesis, which consists of an inner ring element comprising at least one depression for receiving a locking element, wherein the inner ring element is mounted in an outer ring element comprising at least one recess for receiving the locking element, and wherein the locking element is retained in the recess such that it projects into the depression and hence prevents twisting of the inner ring element relative to the outer ring element.

The subject matter of the instant invention is a locking device for use in a prosthesis, in particular for use with a lower leg prosthesis.

From prior art, lower leg prostheses with an artificial knee are known. The artificial knee is fixed to the remaining amputation stump by fixing means, preferably by liner constructions. With such a prosthesis solution the patient is completely mobile and is capable of substantially mastering any kind of movement of a natural knee. The only exception is the movement of the artificial knee, for instance, for crossing the legs when seated, or for Indian style sitting, for instance. This is because it is not possible to make a rotational movement in the lateral plane with conventional artificial knee joints. It appears that this restriction is due to the complexity of the artificial knee joint and is not necessary, either, for the secure course of movement.

As a consequence of this restricted mobility it is, however, not possible for the bearer of the prosthesis to assume, for instance, Indian style sitting or to place the artificial lower leg on the thigh of the other leg. The conventional artificial knee joints are not capable of that since they have not got this degree of freedom. A practical disadvantage consists, for instance, in that the putting on of stockings and shoes due to this restricted mobility is much more complicated for persons with lower leg amputations than for persons without such restriction.

It is therefore an object of the present invention to provide a device with which it is possible to allow an artificial knee joint at least temporarily a further degree of freedom so as to enable a natural course of movement.

This technical object is solved by the locking device according to the invention with the features of claim 1. Advantageous further developments of the present invention are characterized in sub-claims 2 to 6.

The locking device according to the invention for use in a prosthesis consists of an inner ring element comprising at least one depression for receiving a locking element, wherein the inner ring element is mounted in an outer ring element comprising at least one recess for receiving the locking element, and wherein the locking element is retained in the recess such that it projects into the depression and thus prevents twisting of the inner ring element relative to the outer ring element.

Advantageously, the locking element is a cylindrical pin or a ball.

Advantageously, the recess of the outer ring element leads to the depression of the inner ring element on the inner side and is restricted by a locking bolt on the outer side.

Advantageously, the locking bolt is mounted to be movable such that in a first position it fixes the locking element substantially in a first position, and that in a second position the locking element is free to move. In the second position, the locking element is conveyed out of the depression by the introduced rotating movement of the inner ring element, so that the inhibitory effect is completely cancelled. During further rotation of the inner ring element, the locking element cannot fall back into the depression since the latter is no longer aligned with the recess of the outer ring element.

Advantageously, the locking bolt comprises an indentation in which the locking element is received at least partially when the locking bolt is in the second position.

Advantageously, the locking element is, when the locking bolt is in the first position, positioned both in the depression of the inner element and in the recess of the outer ring element so as to prevent a mutual twisting of the two ring elements.

For guaranteeing self-locking it is necessary that the depression in the inner ring element, the recess in the outer ring element, and the indentation in the locking bolt have their geometry coordinated such that there is, on the one hand, no play in the first position and that there is, on the other hand, ensured that for both directions of rotation of the inner ring element the resulting force component acting on the locking bolt acts against the spring force.

In the following, a preferred embodiment of the present invention will be described in detail by means of Figures.

There show:

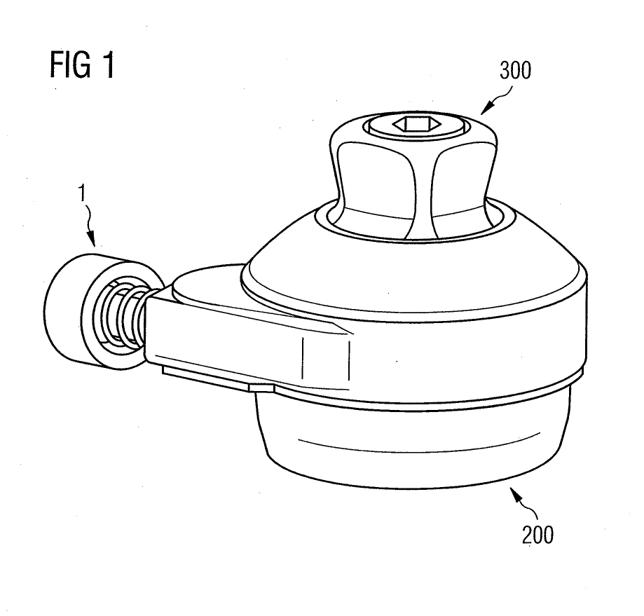

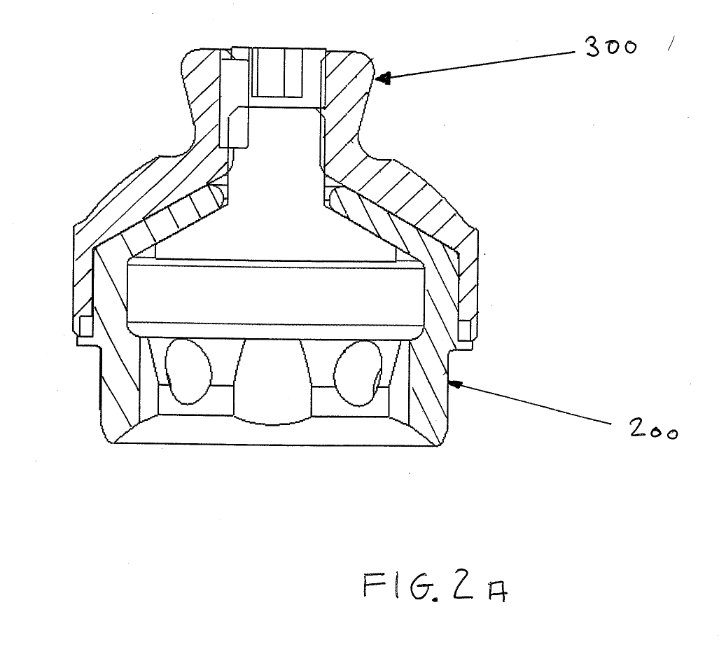

FIG. 1 a perspective illustration of the twist adapter according to the invention;

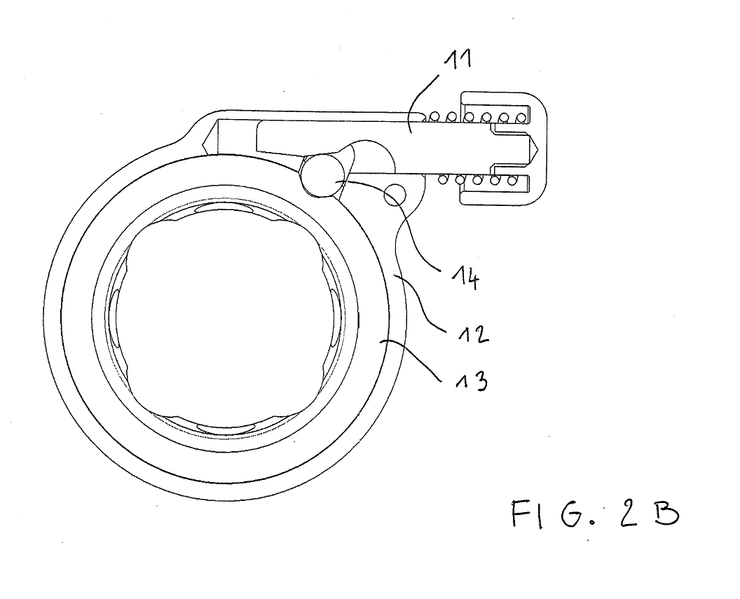

FIG. 2A a sectional view of the twist adapter according to the invention of FIG. 1;

FIG. 2B a plan view of the twist adapter according to the invention of FIG. 1;

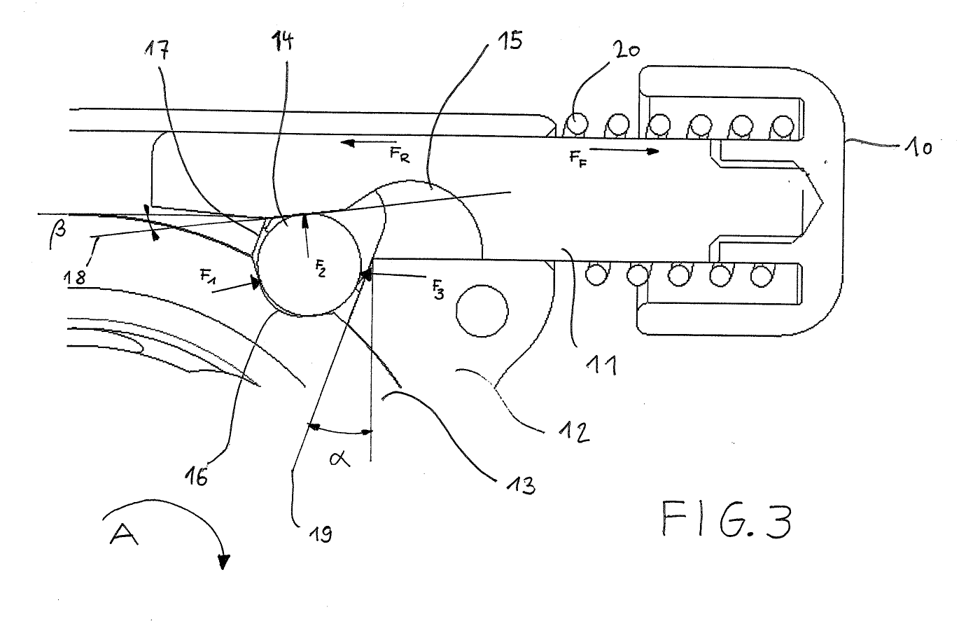

FIG. 3 a systematic sectional view of the twist adapter according to the invention with inhibitory effect in clockwise direction;

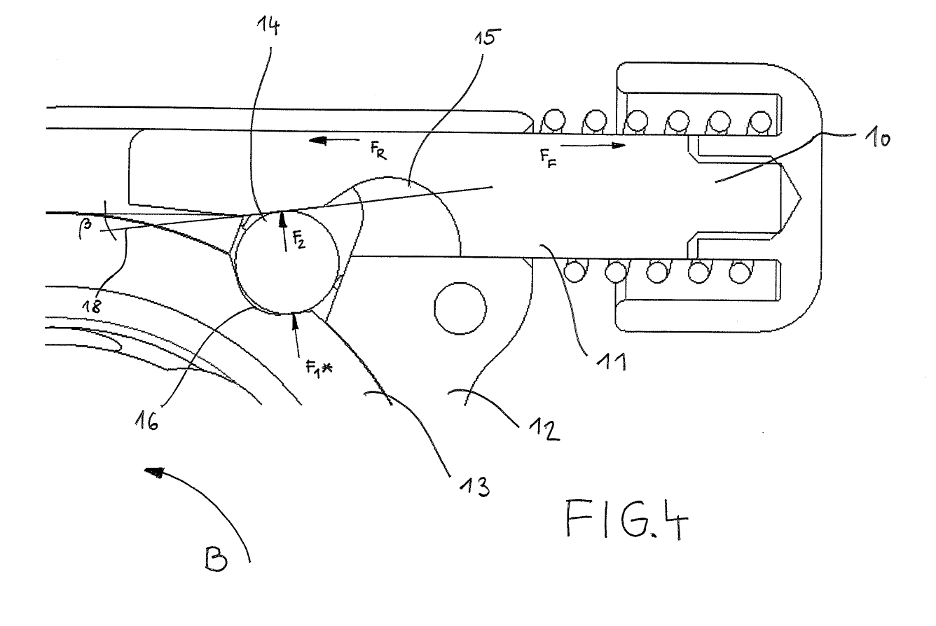

FIG. 4 a sectional view of the twist adapter according to the invention with inhibitory effect in anti-clockwise direction;

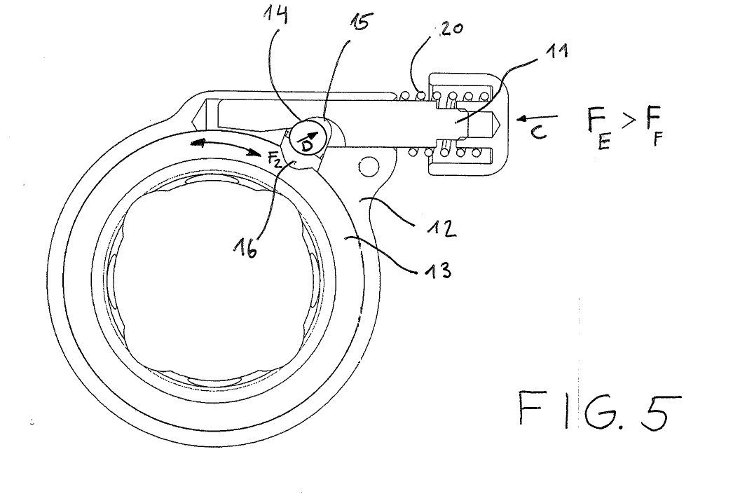

FIG. 5 a sectional view of the twist adapter according to the invention at the time of cancellation of self-locking;

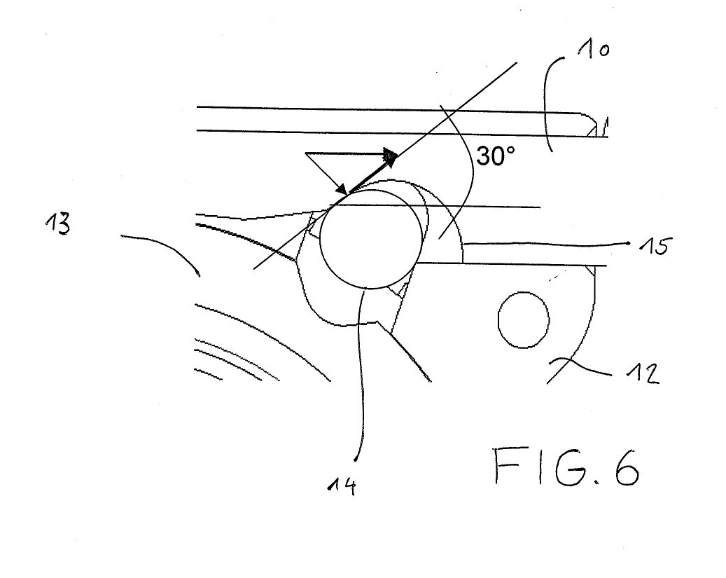

FIG. 6 a schematic detailed illustration of the balance of power pursuant to FIG. 5.

FIG. 1 shows the locking device 1 according to the invention as well as the connecting elements for knee adapters 200 and the connecting element for the shaft 300. Both the connecting element for the shaft 300 and the connecting element for knee adapters 200 are known from prior art and standardized. FIG. 2A shows a sectional view of the locking device according to the invention of FIG. 1.

FIG. 2B illustrates the locking device according to the invention in the locked state, wherein the locking bolt 11 in its rest position contacts the locking element 14 and the locking element 14 remains in the depression in the inner ring element 13. Although the inner ring element 13 and the locking element 14 in FIG. 2B are illustrated such that play might exist, this is just due to the illustration. Expediently, the inner ring element 13 is mounted without play. Advantageously, the bearing of the ring element and the bearing of the locking bolt are designed as sliding bearings, so that, on the one hand, the geometry of construction may be relatively small and, on the other hand, no maintenance of the bearings is required. Due to the sliding bearing geometry it is also possible to design the clearances of the bearings such that splash water and dust protection is ensured.

In the following, the locking mechanism according to the invention will be explained in detail in FIGS. 3 and 4, in particular taking into account the twist direction in clockwise direction and in anti-clockwise direction, and the respective forces will be illustrated.

FIG. 3 shows the locking mechanism according to the invention with a torque A in clockwise direction acting on the inner ring element 13. If a torque A is introduced into the inner ring element 13, a force F₁ acts on the locking element 14 at the contact point in the depression 16 in the inner ring element 13, so that the locking element 14 tries to evade and gets in contact with the outer ring element 12 in the region F₃. The force F₃ constitutes the force component produced by the torque A through the inner ring element 13, the locking element 14, and the entry of forces in the outer ring element 12. The locking element 14 reacts on this introduction of forces by evading in the direction F₂ with the consequence that a force F², is introduced into the locking bolt 11.

Due to the geometry it is not possible that the locking element 14 travels into the depression 15 in the locking bolt 11. In addition to the geometry, the coil spring 20 that introduces a force F_{F} on the locking knob 10 and hence also on the locking bolt 11 hinders the locking bolt 11 from evading in the direction F_{R}. In order to ensure self-locking, a face 18 is arranged at the locking bolt 11 at the angle β which is expediently designed such that the resultant F_{R} with a torque A in clockwise direction points in the direction F_{R}. To this end, it is also necessary that a face at the angle a is provided at the outer ring element 12, which likewise ensures that the resultant comprises a component pointing in the direction F_{R}. Advantageously, the angle a lies between 25 and 35 degrees, especially preferred it is 30 degrees. The angle β advantageously lies between 5 and 10 degrees, preferably it is 7 degrees. The co-ordination of the two angles α and β is expediently performed such that self-locking free of play is achieved.

FIG. 4 illustrates self-locking with a torque that is introduced into the inner ring element 13 and introduces in the point F_{1*} a force on the locking element 14 resting, in the depression 16 in the inner ring element 13. As in the case pursuant to FIG. 3, here there also exists a resultant F_{R} that has to comprise a component in the direction F_{R} to ensure self-locking. For this purpose serves, as already explained in FIG. 3, the use of a face 18 that is preferably arranged at angle of 7 degrees.

FIG. 5 illustrates the point in time at which pressure is exerted on the locking knob 10, with the effect that the coil spring 20 is compressed and the locking bolt II is moved in the direction of the Arrow C. During this movement, a clearance is produced in the region of the locking bolt 11, namely in the indentation 15 in the locking bolt 11, in which the locking element 14 may penetrate. By the movement, the locking element 14, with a simultaneous introduction of torque in the inner ring element 13, is caused to travel out of the depression 16 in the inner ring element 13 in the direction of the Arrow D. As soon as the locking element 14 is no longer positioned in the indentation 16 of the inner ring element, the inner ring element can be rotated freely.

If the depression 16 in the inner ring element 13 is no longer positioned in the region of the locking element 14, the locking element 14 cannot slip back therein anymore and cannot effect any twist inhibition.

Thus, merely a non-recurring introduction of forces F_{E} that is larger than F_{F} is required to overcome the spring force F_{F} and to effect a shifting of the locking bolt 11 in the direction of the Arrow C. With a simultaneous introduction of a torque in the arrow directions (A and B) illustrated into the inner ring element 13, the locking element 14 is conveyed into the indentation 15 in the locking bolt 11, and the inner ring element 13 can be rotated until the depression 16 in the inner ring element 13 is again aligned such that the locking element 14 may engage therein.

FIG. 6 illustrates the forces during the uncoupling of the locking element 14 in the indentation 15 in the locking bolt, so that the inner ring element 13 can now be rotated to move freely.

### LIST OF REFERENCE SIGNS

1 locking device
10 locking button
11 locking bolt
12 outer ring element
13 inner ring element
14 locking element
15 indentation in the locking bolt 11
16 depression in the inner ring element 13
17 recess in the outer ring element 12
18 angle beta at the locking bolt 11
19 angle alpha at the inner ring element 13
20 coil spring
200 connecting element for knee adapter
300 connecting element for shaft

1. A locking device (1) for use in a prosthesis, consisting of an inner ring element (13) comprising at least one depression (16) for receiving a locking element (14), wherein said inner ring element (13) is mounted in an outer ring element (12) comprising at least one recess (17) for receiving said locking element (14), and wherein said locking element (14) is retained in said recess (17) such that it projects into said depression (16) and thus prevents twisting of said inner ring element (13) relative to said outer ring element (12).

2. The locking device (1) according to claim 1, wherein said locking element (14) is a cylindrical pin or a ball.

3. The locking device (1) according to claim 1 or 2, wherein said recess (17) of said outer ring element (12) leads to said depression (16) of said inner ring element (13) on the inner side and is restricted by a locking bolt (11) on the outer side.

4. The locking device (1) according to claim 3, wherein said locking bolt (11) is mounted to be moved such that in a first position it fixes said locking element (14) substantially in a first position, and that in a second position said locking element (14) is free to move.

5. The locking device (1) according to claim 4, wherein said locking bolt (11) comprises an indentation (15) in which said locking element (14) is received at least partially when said locking bolt (11) is in the second position.

6. The locking device (1) according to any of claim 3 or 4, wherein said locking element (14), when said locking bolt (11) is in the first position, is positioned both in said depression (16) of said inner ring element (13) and in said recess (17) of said outer ring element (12), and hence prevents the mutual twisting of said two ring elements (12, 13).
